(19) United States
(12) Patent Application Publication
(10) Pub. No.: US 2013/0207002 A1
(43) Pub. Date: Aug. 15, 2013

(54) LUMINESCENT MATERIAL AND LIGHT EMITTING DEVICE COMPRISING SUCH LUMINESCENT MATERIAL

(71) Applicants: Georg Greuel, Roetgen (DE); Thomas Justel, Witten (DE); Helga Bettentrup, Steinfurt (DE); Julian Plewa, Muenster (DE)

(72) Inventors: Georg Greuel, Roetgen (DE); Thomas Justel, Witten (DE); Helga Bettentrup, Steinfurt (DE); Julian Plewa, Muenster (DE)

(73) Assignee: KONINKLIJKE PHILIPS ELECTRONICS N.V., EINDHOVEN (NL)

(21) Appl. No.: 13/880,079

(22) PCT Filed: Oct. 17, 2011
(86) PCT No.: PCT/IB2011/054589

§ 371 Date: Apr. 18, 2013

(30) Foreign Application Priority Data
Oct. 22, 2010 (EP) 10188525.9

(51) Int. Cl. C09K 11/77; H01J 61/35; A61L 2/10

(52) U.S. Cl. CPC C09K 11/7711; C09K 11/7706; A61L 2/10; H01J 61/35
USPC 250/492.1; 252/301.4 P; 252/301.4 F; 313/486

(57) ABSTRACT

The invention provides a luminescent material comprising a component selected from the group comprising (Y₁₋ₓLuₓ)₉LiSi₆O₂₆:Ln or/and AE₅(PO₄)₃F:Ln,A, wherein Ln is a trivalent rare earth metal, AE is a divalent alkaline earth metal, and A is a monovalent alkaline metal, x>0.0 and <1.0. The luminescent material has an emission peak in the UV-C range when being excited by light in the UV spectrum range. The invention further provides a light emitting device comprising the said luminescent material and a method of using said light emitting device for disinfection or purification of air, water or surfaces.

### FIELD OF THE INVENTION

The present invention relates to luminescent material, especially to the field of luminescent material for light emitting devices emitting UV radiation.

### BACKGROUND OF THE INVENTION

UV radiation sources have found many application areas, such as spectroscopy, cosmetic skin treatment, medical skin treatment, disinfection or purification of water and air, polymer hardening, photochemistry, surface curing, and wafer processing.

Many of the above mentioned application areas require deep UV radiation, i.e. UV-C (200-280 nm) or even VUV radiation (100-200 nm), wherein fast switching cycles and invariance against temperature changes are desired features.

Low-pressure Hg discharge lamps are currently widely used as UV radiation sources and they have an emission spectrum which is dominated by two lines, viz. at 185 and 254 nm. However, increasing the Hg vapor pressure may result in an almost continuous spectrum extending from the deep UV to the deep red spectral range. Moreover, the application of Hg implies a rather strong dependence on temperature and sensitivity to fast switching cycles.

For more than 10 years, the application of dielectric barrier (DB) noble gas excimer discharge has been regarded as an alternative discharge concept for the development of UV emitting radiation sources. The Xe excimer discharge, e.g., emits mainly 172 nm radiation and DB driven quartz lamps comprising Xe as a filling gas show a wall plug efficiency of more than 30%. Quartz lamps based on a Xe excimer discharge are widely used for the cleaning of wafer surfaces due to the sufficiently high energy of the emitted 172 nm (VUV) photons to cleave any type of organic bonds. Fluorescent Xe excimer discharge lamps using one or several VUV to UV-C down-converting phosphors are of particular interest for disinfection or purification purposes.

Presently applied UV luminescent materials for these Xe, Ne, or Xe/Ne excimer lamps still have a couple of drawbacks, for example, including:
low conversion efficiency
low photochemical stability
low chemical stability
low spectral overlap with the germicidal action curve.

Therefore there is a need to develop alternative luminescent materials for converting UV radiation from for instance fluorescent Xe excimer discharge lamps into radiation spectra that can more properly be used for instance in disinfection or purification areas.

### SUMMARY OF THE INVENTION

It is an object of the present invention to provide alternative luminescent materials for light emitting devices emitting UV radiation.

It is another object of the present invention to provide a light emitting device comprising luminescent materials, which device shows intense and efficient UVC emission with a spectral power distribution that fits well to the germicidal action spectrum.

It is yet another object of the present invention to provide a system comprising a light emitting device, which system can use the light emitted by the light emitting device to disinfect or purify air, or water, etc.

According to an embodiment of the present invention, a luminescent material is provided that comprises a component selected from the group comprising (Y₁₋ₓLuₓ)₉LiSi₆O₂₆:Ln or/and AE₅(PO₄)₃F:Ln,A, wherein Ln is a trivalent rare earth metal, AE is a divalent alkaline earth metal, and A is a monovalent alkaline metal, x≧0.0 and ≦1.0.

According to a preferred embodiment, Ln is selected from the group comprising trivalent Pr, Nd or mixtures thereof. AE is selected from the group comprising divalent Ca, Sr, Ba or mixtures thereof. A is selected from the group comprising monovalent Li, Na, K, Rb, Cs or mixtures thereof.

The luminescent material has an emission peak in the UVC (i.e. 200-280 nm) range when being excited by light with an excitation spectrum in the UV spectrum range, preferably in the VUV or UVC range. Light with such an excitation spectrum can be achieved using a Hg or noble gas discharge lamp, for instance, amalgam lamps with an emission peak at around 185 nm, low-pressure Hg discharge lamps with an emission peak at around 254 nm, medium-pressure Hg discharge lamps with an emission peak at around 265 nm, and Xe, Ne, or Xe/Ne excimer lamps with an emission peak at around 172 nm. Alternatively, newly developed LED lamps, like (A1,Ga)N LED lamps, or other types of existing lamps, and even some new types of lamps yet to be developed, can be used as the light source to provide the excitation spectrum, as long as such lamps can emit a proper excitation spectrum needed for the luminescent material to emit the UV-C.

Surprisingly, it has been found that the above proposed luminescent materials show intense and efficient UV-C emission with a spectral power distribution that fits well to the germicidal action spectrum.

According to another embodiment of the present invention, a light emitting device is provided which is capable of emitting a first light in a first UV spectrum range, and comprises at least one of the above proposed luminescent materials to absorb at least part of the first UV light and to emit a second light in a second UV spectrum range different from the first UV spectrum range.

It has been found that such a light emitting device has, for a wide range of applications, especially for germicide application, at least one of the following advantages:
Improved efficacy due to the optimized emission spectrum with respect to the action curve of the application and due to less re-absorption by the luminescent materials;
Improved stability of the UVC output and thus improved operational lifetime of the light emitting device;
smaller dependence of the efficacy on temperature.

According to a preferred embodiment, the light emitting device comprises a discharge lamp provided with a discharge vessel comprising a gas filling having a discharge-maintaining composition, and at least a part of a wall of the discharge vessel is coated with the luminescent material. Alternatively, the discharge lamp comprises a Hg or noble gas discharge lamp. Alternatively, the light emitting device comprises a newly developed LED lamp like a (Al, Ga)N LED lamp, or an already existing lamp type, or even a new type of lamp yet to be developed. For LED lamps, the luminescent materials can be configured as a dome to cover the LED chips, or to be coated on an optical component like a lens or bulb.

According to another embodiment of the present invention, a system comprising at least one of the above proposed light emitting devices is provided, the system further comprising a unit capable of making the light emitted by the light emitting device irradiate an object to be sterilized. This system can be used in germicide applications via photochemical processing with the help of the light emitted by the light emitting device, for instance in disinfection or purification of air, water or surfaces. Such a unit, for example, can be a light guiding means to transport the light from the light emitting device to a surface so that the light can directly irradiate the surface to sterilize said surface. Alternatively, such a unit may comprise a suction device configured to draw certain air into the system so that the light can directly irradiate the air for purification thereof.

According to another embodiment of the present invention, a germicide application method is also provided, which comprises the step of making the light emitted by at least one of the above proposed light emitting devices irradiate an object to be sterilized.

Alternatively, the method can be used in disinfection or purification of air, water or surfaces. Thus, by making the light emitted by the above mentioned light emitting device irradiate air, water or a surface, the air, water or surface can be sterilized.

It has been found that the proposed system and method have a good germicidal effect due to the UV-C emission having a spectral power distribution that fits well to the germicidal action spectrum.

### BRIEF DESCRIPTION OF THE DRAWINGS

The above and other objects and features of the present invention will become apparent from the following detailed description of the various aspects of embodiments with reference to the accompanying drawings.

FIG. 1 shows an XRD pattern of a first exemplary luminescent material according to the present invention (Example I: Ca₅(PO₄)₃F:Pr³⁺ (1%)Na⁺ (1%));

FIG. 2 shows the excitation spectrum (left spectrum), emission spectrum (right spectrum) and reflection spectrum (upper right spectrum) of the first luminescent material according to the present invention (Example I);

FIG. 3 shows a comparison between the emission spectrum of the first luminescent material (Example I) and the desired spectrum of the germicidal action;

FIG. 4 shows an XRD pattern of a second exemplary luminescent material according to the present invention (Example II: Sr₅(PO₄)₃F:Pr³⁺ (1%)Na⁺ (1%));

FIG. 5 shows the excitation spectrum (left spectrum), the emission spectrum (right spectrum) and the reflection spectrum (upper right spectrum) of the second luminescent material according to the present invention (Example II);

FIG. 6 shows a comparison between the emission spectrum of the second luminescent material (Example II) and the desired spectrum of the germicidal action curve;

FIG. 7 shows an XRD pattern of a third exemplary luminescent material according to the present invention (Example III: Y₉LiSi₆O₂₆:Pr³⁺ (1%));

FIG. 8 shows the excitation spectrum (left spectrum), the emission spectrum (right spectrum) and the reflection spectrum (upper right spectrum) of the third luminescent material according to the present invention (Example III);

FIG. 9 shows an XRD pattern of a fourth exemplary luminescent material according to the present invention (Example IV: Ba₅(PO₄)₃F:Pr³⁺ (1%)Na⁺ (1%));

FIG. 10 shows the excitation spectrum (left spectrum), the emission spectrum (right spectrum) and the reflection spectrum (upper right spectrum) of the fourth luminescent material according to the present invention (Example IV).

### DETAILED DESCRIPTION OF EMBODIMENTS

The detailed description of the embodiments given below will mainly focus on examples of luminescent materials. As for the light emitting device, the system and the method proposed in the present invention, the prior part has given a useful description and reference can be made to the existing relevant papers or products.

### Example I

Example I refers to Ca₅(PO₄)₃F:Pr³⁺ (1%)Na⁺ (1%), which can be made in the following way:

The starting materials 1.009 g CaCO₃, 4.0004 g CaHPO₄.2H₂O, 0.32 g nanoscale CaF₂, and 0.076 g PrF₃ and 0.016 g NaF have been milled for 0.5 hours. The blend has been subsequently annealed at around 1100° C. under Nitrogen for 1 hour. Finally, the material is milled and sieved through a 36 μm sieve.

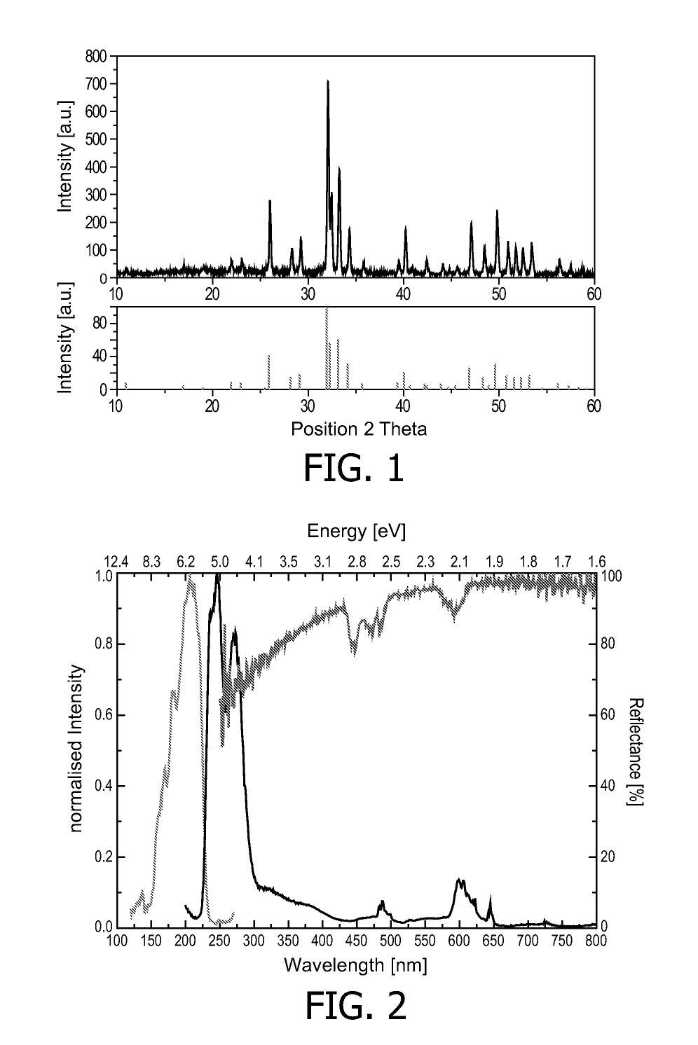

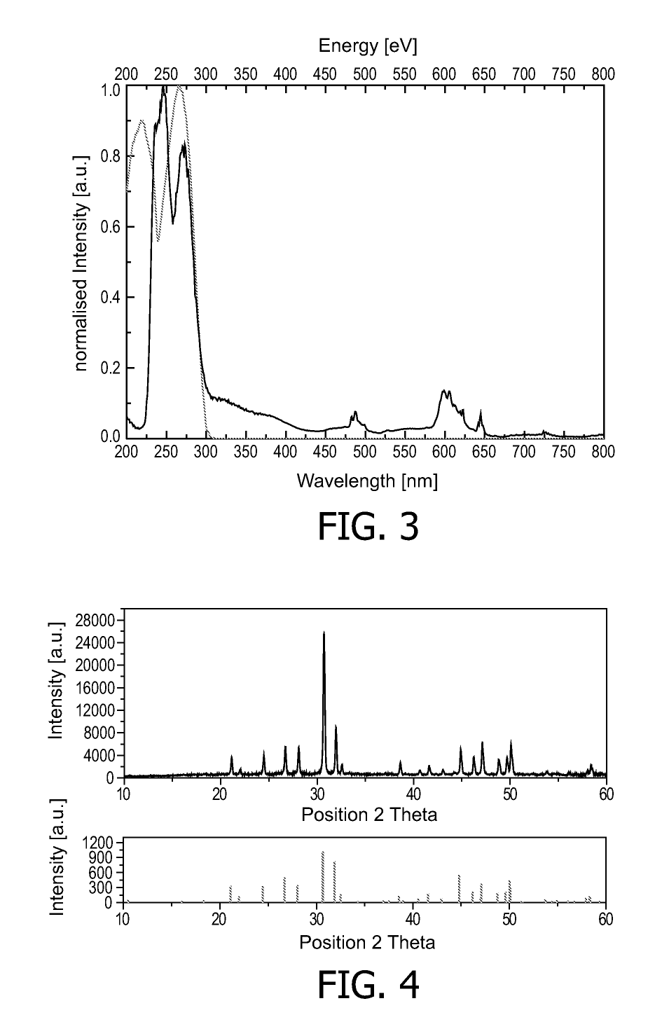

FIG. 1 shows an XRD pattern of the material of Example I. FIG. 2 shows the excitation spectrum (left spectrum), the emission spectrum (right spectrum) and the reflection spectrum (upper right spectrum) of the material of Example I. FIG. 3 shows a comparison between the emission spectrum (the curve with relatively narrow extension along the wavelength in the drawing, as well as in other drawings of the same type referred to below) of the material of Example I and the desired spectrum of the germicidal action. The emission maximum of Ca5(PO4)3F:Pr,Na is at around 245 nm, which shows a good overlap with the germicidal action curve. It can clearly be seen that this material is an excellent material for use in discharge lamps for UV-C radiation.

### Example II

Example II refers to Sr₅(PO₄)₃F:Pr³⁺ (1%)Na⁺ (1%), which can be made in the following way:

The starting materials 5.036 g SrCO₃, 2.675 g (NH₄)₂HPO₄.2H₂O, 0.487 g nanoscale SrF₂, and 0.076 g PrF₃ and 0.016 g NaF have been milled for 0.5 hours. The blend has been subsequently annealed at around 1100° C. under Nitrogen for 1 hour. Finally, the material is milled and sieved through a 36 μm sieve.

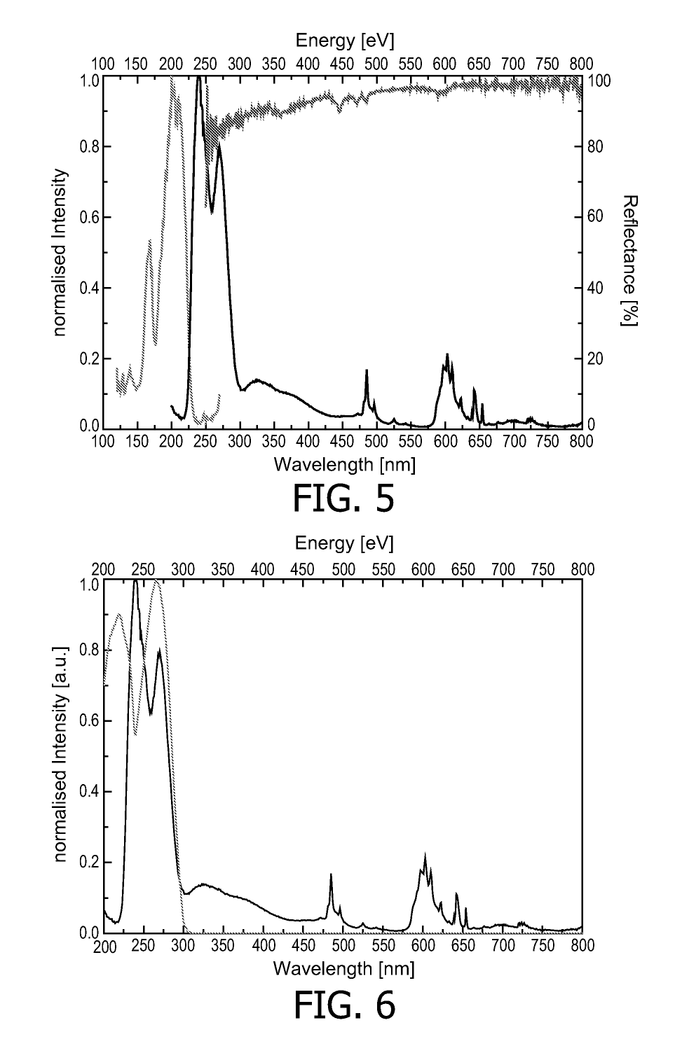

The emission maximum of Sr₅(PO₄)₃F:Pr,Na is at about 240 nm, which also shows a good overlap with the germicidal action curve. It can clearly be seen from FIGS. 4-6 that this material is an excellent material for use in discharge lamps for UV-C radiation.

### Example III

Example III refers to Y₉LiSi₆O₂₆:Pr³⁺ (1%), which can be made in the following way:

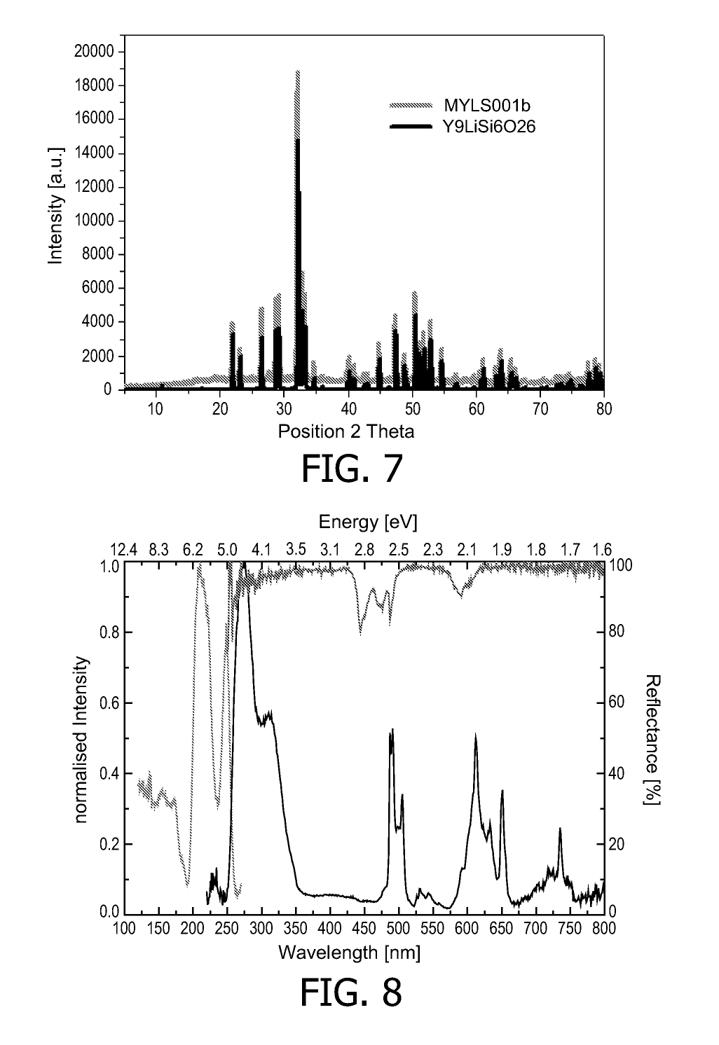

The starting materials 4.000 g Y₂O₃, 0.147 g Li₂CO₃, 1.433 g nanoscale SiO₂, and 0.061 g Pr₆O₁₁ are suspended in ethanol and the material is ground until the solvent has completely evaporated. Afterwards, the dried material is fired at 1000° C. under CO for 6 hours and subsequently ground and fired at 1100° C. under CO for 6 hours. Finally, the material is milled and sieved through a 36 μm sieve. It can clearly be seen from FIGS. 7-8 that this material is an excellent material for use in discharge lamps for UV-C radiation.

### Example IV

Example IV refers to Ba₅(PO₄)₃F:Pr³⁺(1%)Na⁺(1%), which can be made in the following way:

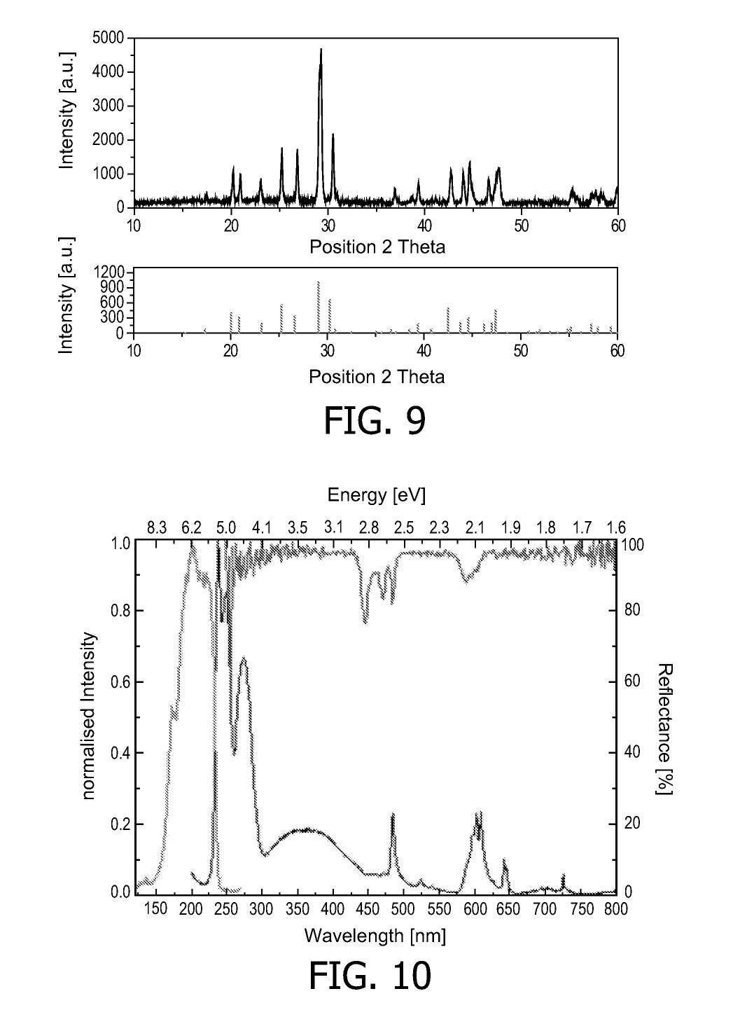

The starting materials 5.036 g BaCO₃, 2.675 g (NH₄)₂HPO₄.2H₂O, 0.487 g nanoscale BaF₂, and 0.076 g PrF₃ and 0.016 g NaF have been milled for 0.5 hours. The blend has been subsequently annealed at 1100° C. under Nitrogen for 1 hour. Finally, the material is milled and sieved through a 36 μm sieve. It can clearly be seen from FIGS. 9-10 that this material is an excellent material for use in discharge lamps for UV-C radiation.

The embodiments described above are merely preferred embodiments of the present invention. Other variations of the disclosed embodiments can be understood and effected by those skilled in the art in practicing the claimed invention, from a study of the drawings, the disclosure, and the appended claims. These variations shall also be considered to be within the scope of the present invention. In the claims and description, use of the verb “comprise” and its conjugations does not exclude other elements or steps, and the indefinite article “a” or “an” does not exclude a plurality.

1. A luminescent material comprising a component selected from the group comprising (Y₁₋ₓLuₓ)₉LiSi₆O₂₆:Ln or/and AE₅(PO₄)₃F:Ln,A, wherein Ln is a trivalent rare earth metal, AE is a divalent alkaline earth metal, and A is a monovalent alkaline metal, x≧0.0 and ≦1.0.

2. (canceled)

3. (canceled)

4. (canceled)

5. (canceled)

6. (canceled)

7. (canceled)

8. (canceled)

9. (canceled)

10. (canceled)

11. The luminescent material according to claim 1, wherein the luminescent material has an emission peak in the UVC range when being excited by light with an excitation spectrum in the UV spectrum range.

12. A light emitting device, which is capable of emitting a first light in a first UV spectrum range, comprising a luminescent material according to claim 1 to absorb at least part of the first UV light and to emit a second light in a second UV spectrum range different from the first UV spectrum range.

13. The light emitting device according to claim 12, wherein the light emitting device comprises a discharge lamp, provided with a discharge vessel comprising a gas filling with a discharge-maintaining composition, and at least a part of a wall of the discharge vessel is coated with the luminescent material.

14. The light emitting device according to claim 12, wherein the discharge lamp comprises a Hg or noble gas discharge lamp, or/and a LED lamp.

15. A system comprising a light emitting device according to claim 12, the system further comprising a unit capable of making the light emitted by the light emitting device irradiate an object to be sterilized.

16. A method of applying a germicidal agent, comprising the step of making the light emitted by a light emitting device according to claim 12 irradiate an object to be sterilized.
